(19) United States
(12) Patent Application Publication
(10) Pub. No.: US 2005/0203554 A1
(43) Pub. Date: Sep. 15, 2005

(54) Corneal marking device, and method of corneal marking

(76) Inventor: Ronald E. Dykes, The Woodlands, TX (US)

Correspondence Address: LAW OFFICES OF WILLIAM L. KLIMA, P.C., 2046-C JEFFERSON DAVIS HIGHWAY, STAFFORD, VA, 22554, US

(21) Appl. No.: 10/796,491

(22) Filed: Mar. 10, 2004

(51) Int. Cl. A61B 18/18

(52) U.S. Cl. 606/166

(57) ABSTRACT

A corneal marking device configured to indent and mark the surface of the cornea of the eye. Preferably, the indentation and marking are temporary in nature. Further, a method of marking the cornea of the eye.

### FIELD OF THE INVENTION

The present invention relates to a medical device for marking the cornea of an eyeball, and method of cornea marking.

### BACKGROUND OF THE INVENTION

In the field of surgical refractive correction of the eye, there exist a number of surgical procedures for improving or correcting vision. A particular popular refractive correction procedure today is LASIK, involving cutting a corneal flap and then using a laser to reshape the cornea of the eye. A lesser-known but upcoming surgical procedure for refraction correction of the eye is known as conductive keratoplasty.

Conductive keratoplasty involves applying a conductive keratoplasty tip at specific positions in a particular pattern on the surface of the cornea, or slightly into the surface of the cornea (e.g. 460 microns plus or minus 10 microns), to reshape the cornea and correct and improve vision. In order to carry out this particular surgical procedure, the surface of the cornea must be carefully marked to allow an eye surgeon to accurately and precisely place the conductive keratoplasty tip at specific points in the surface of the cornea, again typically in a specific pattern.

The current procedure utilizes a corneal printer made of autoclavital metal (i.e. stainless steel) having one or more raised printing surfaces to print in a particular pattern. The raised printing surfaces are substantially flat printing surfaces, and are configured to apply a tissue marking ink or dye in a particular pattern on the surface of the cornea. Specifically, ink or dye is applied to the printing surfaces (e.g. using an ink or dye pad), and then the corneal marking device is applied to the cornea. The ink or dye is transferred from the printing surfaces onto the surface of the cornea to print a pattern of ink or dye onto the surface of the cornea.

The current corneal printing device being made of metal, is not transparent making it somewhat difficult to properly center and accurately and precisely apply the printing end of the device onto the cornea of the eye. Further, the current cornel printing device is limited in application, since it can only print on the cornea when used in conjunction with a tissue marking dye or ink.

The cornel dimpling and marking device according to the present invention provides a number of substantial advantages and improvements over the current corneal printing device and printing method available.

### SUMMARY OF THE INVENTION

A first object of the present invention is to provide an improved corneal marking device.

A second object of the present invention is to provide an improved corneal printing device.

A third object of the present invention is to provide an improved corneal marking device having a template end portion.

A fourth object of the present invention is to provide an improved corneal marking device having a template end portion made of a transparent material.

A fifth object of the present invention is to provide an improved corneal marking device, including at least one protrusion configured to temporarily indent, preferably dimple, and mark at least one surface position on the cornea by application of pressure from a tip of the protrusion onto the corneal surface of the eye.

A sixth object of the present invention is to provide an improved corneal marking device configured to accurately mark positions on the surface of the cornea in a highly reproducible manner from procedure to procedure, and from patient to patient.

The present invention is directed to a corneal marking device, and method of marking a cornea of an eye.

For purposes of the present invention, the cornea of the eye can be that of a human eye or animal eye.

The corneal marking device according to the present invention can be made entirely, or portions thereof, from a wide variety of materials, including plastic, metal, composite, glass, ceramic, or other suitable materials. The corneal marking device according to the present invention is preferably made from a plastic material, in particular a transparent plastic material and configured to allow an eye surgeon to view through the template or marking end portion of the device during use. Preferred plastics include poly carbonate, poly styrene, poly theromide resins, or other suitable plastic resin blends. Preferably, the corneal marking device according to the present invention is disposable, and for one time use. The corneal marking device according to the present invention is preferably made by a plastic injection molding process using a carefully machined and highly accurate and precise mold cavity to provide high accuracy, and reproduce ability of corneal marking results from procedure to procedure and eye to eye.

The corneal marking device according to the present invention is provided with one or more protrusions configured to make a temporary indent, preferably a temporary dimple, and provide temporary markings or a pattern of markings on the surface of the cornea by application of pressure. For example, the tips of the protrusions are configured (e.g. contoured, shaped, textured and/or finished) so that when pressure is applied to the corneal marking device the protrusions of the corneal marking device press against the surface of the eye. The tips of the protrusions make indents, preferably dimples, that temporary mark the surface of the eye. The indenting or dimpling process changes the angle of reflection of light hitting the indents or dimples verses the surrounding tissue, which can be visually detected by the eye surgeon, especially with magnified vision using a loop or microscope. In a short amount of time (e.g. less than 10 minutes or even less than 4 minutes), these indents or dimples dissipate (i.e. heal) and the markings created thereby visually disappear.

The tips of the protrusions are preferably shaped, contoured, textured and/or finished so as not to penetrate, cut, scratch, or otherwise compromise or damage the surface of the cornea, but instead only provide temporary marks on the surface of the cornea. Specifically, in some embodiments, the protrusions are configured so that the marks last approximately less than 10 minutes from the time of the application of pressure from the protrusions against the surface of the cornea of the eye. This allows enough time for the eye surgeon to conduct the surgical procedure such as conductive keratoplasty.

In preferred embodiments, the protrusions are conical-shaped structures having rounded end tip portions. The surface is provided with a smooth texture. The exact sharpness and curvature of the tip portions of the protrusions are carefully designed or selected so as to not penetrate (or substantially penetrate), cut, scratch, damage or otherwise compromise the surface of the cornea of the eye when applied under pressure.

Preferred embodiments of the corneal marking device according to present invention utilize a plurality of protrusions arranged in a particular pattern. In a preferred embodiment, multiple sets (e.g. eight (8) sets) of three (3) protrusions are provided on a ring-shaped template end portion of the corneal marking device. The sets of three (3) protrusions are each oriented along radii extending from the center of the cornea (i.e. center of pupil). Further, for example, the multiple sets of three (3) protrusions are equally spaced apart around an arc disposed within the dimensions of the cornea, and centered off the center of the cornea. Further, the individual respective protrusions of the multiple sets of three (3) protrusions are located on three (3) separate arcs disposed within the dimensions of the cornea, which arcs are centered off the center of the cornea. The three (3) separate arcs are located at three (3) different radius (e.g. 6 mm, 7 mm and 8 mm) from the center of the cornea. Additional single protrusions can be provided between these sets of three (3) protrusions to provide incremental markings to 1) facilitate marking for astigmatic correction; and 2) allow the eye surgeon to more accurately judge distances between the sets of three (3) protrusions for placement of the conductive keratoplasty tip during conductive keratoplasty.

The protrusions can be uniform in size, shape, design, texture, finish or otherwise confirmation (e.g. macro and micro conformation), or alternatively, can be different (e.g. different conformations, sizes and/or shapes). For example, the protrusions can be varied to change the size, shape, depth of the indentations or depressions (e.g. preferably dimples) made in the surface of the cornea. For example, instead of round-shaped dimples, triangle-shaped, square-shaped, cross-shaped temporary indentations or depressions can be made in the surface of the cornea. However, round spherical-shaped dimple type indentations or depressions are preferably made, since light impinging on the spherical-shaped indentations or depressions reflect towards the center of the dimples and cancels out by light wave interference effect creating visual markings or aberrations on the surface of the cornea.

### BRIEF DESCRIPTION OF THE DRAWINGS

FIG. 1 is a prospective view of an embodiment of the corneal marking device according to the present invention.

FIG. 2 is a top planar view of the corneal marking device shown in FIG. 1.

FIG. 3 is a bottom planar view of the corneal marking device shown in FIG. 1.

FIG. 4 is a side elevational view of corneal marking device shown in FIG. 1, being applied to the surface of the cornea of an eyeball.

FIG. 5 is a partial broken away top detailed perspective view of the ring-shaped template end portion of the corneal marking device shown in FIG. 1.

FIG. 6 is a partial broken away bottom detailed prospective view of the ring-shaped template end portion of the corneal marking device shown in FIG. 1.

FIG. 7A is a partial broken away top detailed planar view of the ring-shaped template end portion of the corneal marking device shown in FIG. 1.

FIG. 7B is a partial broken away bottom detailed planar view of the ring-shaped template end portion of the corneal marking device shown in FIG. 1.

FIG. 8 is a detailed vertical center cross-sectional view along a length of the ring-shaped template end portion of the corneal marking device, as indicated in FIG. 7B.

FIG. 9 is a detailed vertical center cross-sectional view of a protrusion of the corneal marking device shown in FIG. 1.

FIG. 10 is a top planar view of an eyeball showing the cornea marked in a pattern by use of the cornel marking device according to the present invention.

### DETAILED DESCRIPTION OF PREFERRED EMBODIMENTS

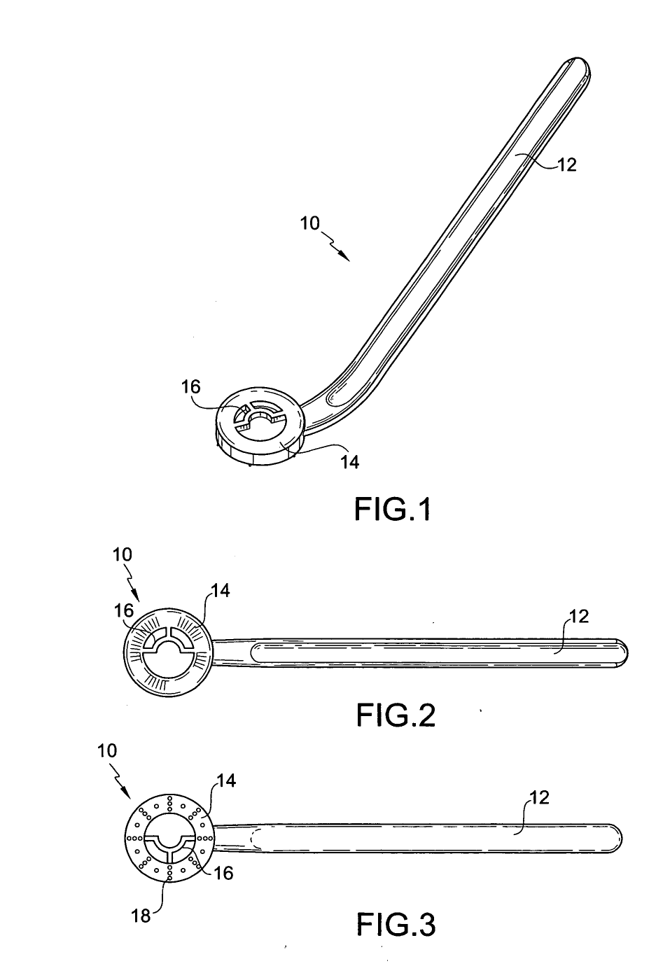

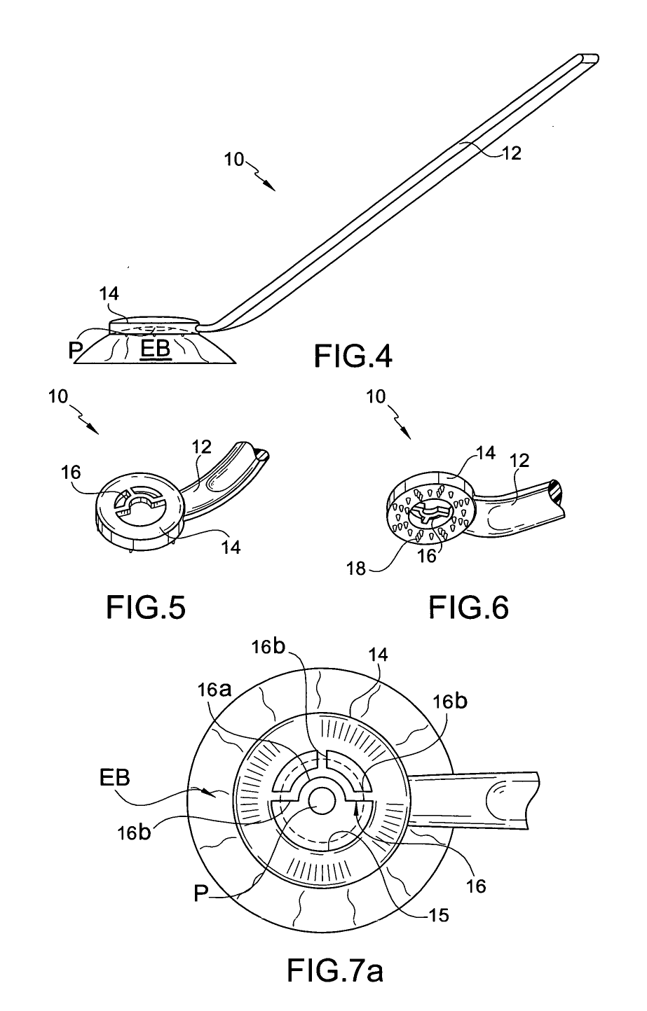

A preferred embodiment of the corneal marking device 10 according to the present invention is shown in FIG. 1.

The corneal marking device 10 includes a handle portion 12 connected to a ring-shaped template end portion 14. Preferably, handle portion 12 and ring-shaped template end portion 14 are made as a single piece, however, the handle portion 12 and ring-shaped template end portion 14 can be made as separate components or parts, and then connected or assembled together.

The corneal marking device 10 is preferably made of a transparent or see-through plastic material such as polycarbonate, polystyrene or poly theromide resin(s). Further, the corneal marking device 10 is preferably made by an injection molding process in a highly controlled manner so that the device 10 is highly accurate, highly precise, and provides surgical results that are highly reproducible from device to device, procedure to procedure, and eye to eye. More specifically, the use of plastic injection molding for manufacturing the device allows for a mold cavity to be machined, wired and/or polished to a very high degree of precision and accuracy regarding the conformation of the protrusions, in particular the conformation of the protrusion tips (e.g. design, shape, contour, texture, finish), spacing, and location of the tips of the protrusions in three-dimensions (3-D). Carefully selecting the particular type(s), blend(s) and/or additives of the plastic resin and highly controlling the plastic injection molding parameters (e.g. temperature, pressure, flow rate, mold temperature) allows for the production of highly precise and accurate molded parts that are highly dimensionally stable within a reasonable temperature range (e.g. suitable ambient temperature range) and are suitably shelf stable (e.g. one (1) to two (2) years).

The ring-shaped template end portion 14 is preferably provided with a centered through-hole 15 as shown. Alternatively, the through-hole 15 can be eliminated, and the template end portion can be made as a solid round template portion using see-through plastic material to provide a see-through window in the center thereof. However, the open design of the ring-shaped template portion 14 having the center through-hole 15 allows for direct unimpeded viewing of the corneal surface of the eyeball through the through-hole 15 by the eye surgeon to assist in precisely and accurately positioning and centering of the ring-shaped template end portion 14 on the cornea. Again, the transparent nature of the plastic material used for making the ring-shaped template end portion 14 also allows for good to excellent viewing through the thickness of the ring-shaped template portion 14 itself. However, there may be some slight visual distortion, magnification, or visual impairment when viewing through the ring-shaped template end portion 14 itself due to the curvature of the top and bottom surfaces of the ring-shaped template end portion 14 combined with the particular index of refraction and degree of surface reflectance of the plastic material selected.

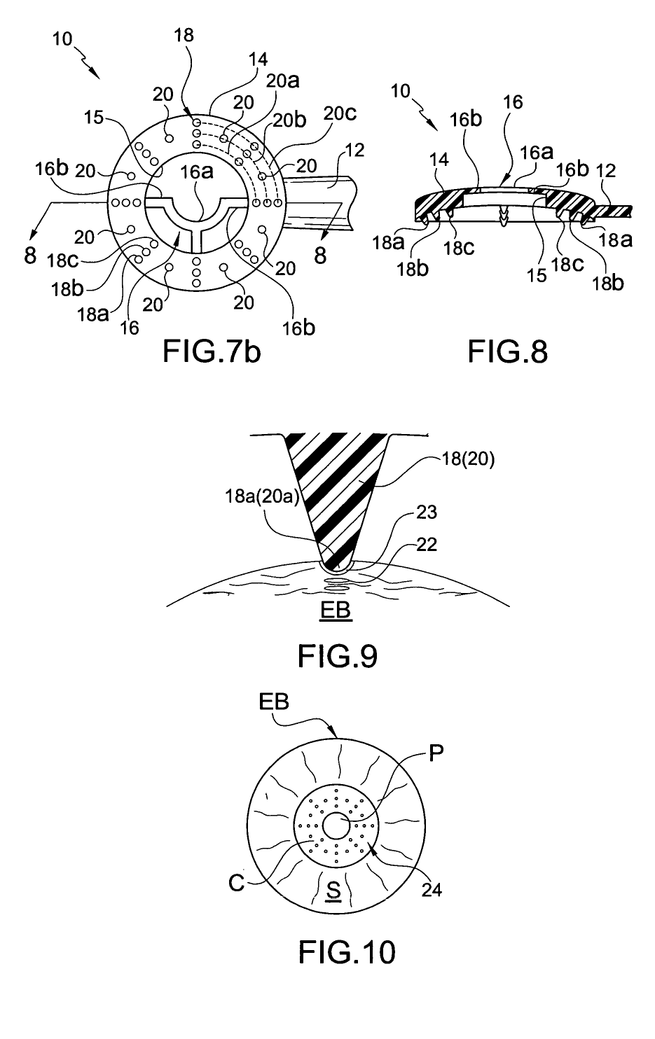

The ring-shaped template end portion 14 is provided with a sight 16. The sight 16 includes a one-half circular sight portion 16A supported by three (3) spoke sight portions 16B, 16B, 16B, as shown in FIG. 7A. The inner surface of the one-half circular sight portion 16A is. shaped (e.g. half circle) to be aligned and partially surround the pupil P of the eyeball EB as shown in FIG. 7A. In this manner the ring-shaped template end portion 14 is centered on the cornea C.

The ring-shaped template end portion 14 is provided with a plurality of protrusions 18. A preferred pattern of protrusions 18 is shown in FIG. 7B. In this pattern, eight (8) sets of three (3) protrusions 18a, 18b, 18c are orientated along eight (8) equally spaced apart radii extending from the center of the ring-shaped template portion 14 to coincide with the center of the cornea C. These sets of three (3) protrusions, 18a, 18b, 18c are equally spaced around the three (3) separate arcs 20a, 20b, 20c. The arcs 20a, 20b, 20c are equally spaced apart from each other, and are located at different length radius from the center of the ring-shaped template end portion 14. For example, the outer arc 20a has a radius of 8 millimeters (mm), the middle arc 20b has a radius of 7 millimeters (mm) and the inner arc 20c has a radius of 6 millimeters (mm). Each set of three (3) protrusions 18a, 18b, 18c define a line segment along the length of each radii at eight separate locations. Additional single protrusions 20 bisect the distance between adjacent sets of three (3) protrusions 18a, 18b, 18c are located alone the middle arc 20b. These additional protrusions 20 help the eye surgeon to correct astigmatic vision, and locate and judge specific positions between the adjacent sets of three (3) protrusions 18, 118b, 118c for proper placement of the conductive keratoplasty tip during conductive kertoplasty.

The protrusions 18, 20 have tip portions 118a, 20a configured to apply pressure onto the surface of the cornel at specific point positions. The tip portions 118a, 20a of the protrusions 18, 20, respectively, are shaped to be somewhat pointed, however, somewhat rounded at the very end thereof so as to not penetrate, puncture, cut, scrape, damage or otherwise compromise the surface of the cornea. However, when pressure is applied, the protrusions 18, 20 contact the surface of the cornea and create temporary dimples 23 (See FIG. 9) that provide temporary visual marks, which disappear with time (e.g. typically under 10 minutes). Specifically, when pressure is applied by the tip portions of the protrusions onto to the surface of the cornea, small dimples 23 are made in the surface of the cornea. These small dimples 23 change the angle of reflectance of light impinging on the surface of the cornea verses the surrounding corneal tissue, which can be visually detected by the aided or unaided eye. These dimples 23 slowly rebound or heal and the coincident marks disappear within approximately ten minutes in time. Afterwards, the dimples 23 and coincident marks disappear and can no longer be viewed.

In use, an eye surgeon grabs the handle portion 12 of the corneal marking device 10 with his or her hand. The eye surgeon centers the ring-shape template end portion 14 of the cornel marking device 10 above the cornea by viewing through the sight 16. Specifically, the ring-shaped template end portion is moved around and adjusted by the handle portion 12 so that pupil is centered within the half circle sight portion 16a. The eye surgeon makes sure that the handle of the device 10 is set at the proper orientation and angle and that the ring-shaped template end portion 14 is centered on the cornea. Then, the eye surgeon then applies the ring-shaped template end portion 14 against the surface of the cornea with the protrusions 18, 20 making contact therewith. Additional pressure is applied by the eye surgeon's hand to force the ring-shaped template end portion 14 further towards the surface of the eye so that the tip portions 118a, 20a of protrusions 18, 20, respectively, exert enough pressure so as to cause temporary dimpling and marking of a pattern on the surface of the cornea. The device 10 is then removed, and then the cornea marked with the particular pattern is now ready for the surgical procedure elected (e.g. conductive keratoplasty).

The above procedure can be accomplished without the use of tissue dye or ink. However, visually enhancement of the marks can be accomplished by evenly applying tissue dye or ink (e.g. by dye or ink pad) to the tip portions 18a, 20a of the protrusions 18, 20, respectively. In this manner, both dimpling in combination with dying or inking enhances the visually acuity of the marks made by the device 10. Both the dimples and ink or dye are temporary, and will dissipate and disappear relatively quickly.

The method according to the present invention involves marking the cornea of the eye with at least one mark, or preferably a pattern of marks.

The method includes the step of making a mark by application of force or pressure against the surface of the cornea. In a preferred embodiment, the mark is made by application of a force, in particular a concentrated force (e.g. point force), or concentrated pressure against the surface of the cornea at a location where a mark is desired. This is preferably accomplished by a corneal marking device having at least one protrusion. The protrusion is configured to apply a concentrated forces such as a point force or pressure against the surface of the cornea. In a preferred embodiment, the tip of the protrusion is configured (e.g. shaped, contoured, textured, finished) so at to make a slight indentation or depression into the surface of the cornea, preferably a dimple-shaped indentation or depression. The indentation or depression is created by compressing the tissue at a point with the tip of the protrusion. This causes elastic and/or plastic type deformation of the affected tissue. The dimple will remain for only a short period of time (e.g. up to 10 minutes) as the tissue elastically or plastically rebounds dissipating and finally eliminating the dimple-shaped indentation or depression.

The temporary indentation or depression, in particular a dimple-shaped indentation or depression, causes light impinging on the surface of the dimple to reflect towards the center of the dimple-shaped depression and cancel out due to light wave interference resulting in what appears to be a marking or visual aberration on the surface of the cornea. This mark can be easily visually detected with high precision and accuracy by the unaided eye, and in particular when viewing with an aided eye (e.g. through a loop at 10x or greater).

In a preferred method according to the present invention, a pattern of marks can be made according to the above described manner allowing for use of the method to mark a particular pattern of marks on the cornea of the eye to allow a surgical procedure (e.g. conductive keratoplasty). In a further preferred embodiment, the marks made by this method can be visually enhance by adding the step of using tissue dye or ink on the tips of the protrusions to cause marking by both dimpling and coloring effects.

1. A corneal marking device configured for indenting and marking a cornea of an eye in a particular patter, said device comprising:
a handle portion;
a template portion connected to said handle portion, said template portion configured to provide a pattern of indentations and marks on a corneal surface of an eye.

2. A device according to claim 1, wherein said template portion is a ring-shaped template portion.

3. A device according to claim 1, wherein said template portion is provided with at least one protrusion configured to make an indent and mark on the surface of the cornea of the eye when pressure is applied by said at least one protrusion against the corneal surface of the eye.

4. A device according to claim 2, wherein said ring-shaped template portion is provided with at least one protrusion configured to indent and make a mark on the corneal of the eye when pressure is applied by said at least one protrusion against the corneal surface of the eye.

5. A device according to claim 2, wherein said ring-shaped template portion is configured to facilitate positioning of said ring-shaped template portion relative to the cornea of the eye.

6. A device according to claim 5, wherein said ring-shaped template portion is provided with at least one sight to facilitate position of said ring-shaped template portion relative to the cornea of the eye.

7. A device according to claim 6, wherein said at least one sight is defined by a half-circle edge centered in a centered through hole in said ring-shaped template portion.

8. A device according to claim 1, wherein said at least one protrusion is a single protrusion, and said device is configured to apply a pattern of marks on the surface of the cornea of the eye by multiple applications of said single protrusion against the surface of the cornea of the eye.

9. A device according to claim 1, wherein said at least one protrusion is defined by a plurality of protrusions set in a particular pattern.

10. A device according to claim 9, wherein said device is configured to apply a full pattern of marks on the surface of the cornea of the eye by a single application of said protrusions against the surface of the cornea of the eye.

11. A device according to claim 2, wherein said at least one protrusion is a plurality of protrusions.

12. A device according to claim 11, wherein said plurality of protrusions are spaced apart and located on at least one concentric arc of said ring-shaped template portion.

13. A device according to claim 11, wherein said plurality of protrusions are spaced apart and located on at least one radius of said ring-shaped template portion.

14. A device according to claim 11, wherein said plurality of protrusions are spaced apart and located on at least one concentric arc and on at least one radius of said ring-shaped template portion.

15. A device according to claim 12, wherein said plurality of protrusions are equally spaced apart and located on at least one concentric arc of said ring-shaped template portion.

16. A device according to claim 13, wherein said plurality of protrusions are equally spaced apart and located on at least one radii of said ring-shaped template portion.

17. A device according to claim 14, wherein said plurality of protrusions are equally spaced apart and located on at least one concentric arc of said ring-shaped template portion, and said plurality of protrusions are equally spaced apart and located on at least one radii of said ring-shaped template portion.

18. A device according to claim 17, wherein said plurality of protrusions are provided in a pattern of separate sets of three protrusions located on multiple equally spaced apart radii of said ring-shaped template portion.

19. A device according to claim 18, wherein additional single protrusions are equally spaced between said sets of three protrusions.

20. A device according to claim 19, wherein said additional single protrusions are located on a same medial arc of said ring-shape template end portion, and a center protrusion of each said separate sets of three protrusions are also located on said medial arc of said ring-shaped template end portion.

21. A method of marking a cornea of an eye, said method comprising the steps of:
applying an indentation creating a marking on a surface of a cornea of an eye using a corneal marking device having at least one protrusion.

22. A method according to claim 21, wherein said pattern is applied by said device having multiple protrusions.

23. A method according to claim 21, wherein said indentation and marking are temporary.
